(19) United States
(12) Patent Application Publication
(10) Pub. No.: US 2022/0057384 A1
(43) Pub. Date: Feb. 24, 2022

(54) SLEEP MODULATION AGENT

(71) Applicant: Oxford University Innovation Limited, Oxford (Botley) (GB)

(72) Inventors: Gero MIESENBOECK, Oxford (Oxfordshire) (GB); Anissa KEMPF, Oxford (Oxfordshire) (GB); Seoho SONG, Oxford (Oxfordshire) (GB)

(21) Appl. No.: 17/416,592

(22) PCT Filed: Oct. 19, 2019
(86) PCT No.: PCT/GB2019/053622

§ 371 Date: Jun. 21, 2021

(30) Foreign Application Priority Data
Dec. 21, 2018 (GB) 1821066.6

(51) Int. Cl. G01N 33/50; A61K 49/00

(52) U.S. Cl. CPC G01N 33/5035; A61K 49/0008; G01N 33/5058

(57) ABSTRACT

The present invention relates to one or more ligands of a potassium channel β subunit for use in therapy, and in particular for use in treating or preventing a sleep disorder in a subject. The invention also provides a method of screening a test compound to determine if it is a substrate of a potassium channel β subunit.

The invention relates to agents for use in the modulation of sleep, in particular, to the use of such agents for the treatment of sleep disorders.

Sleep disturbances are among the most common medical problems. A large majority (75%) of adults in Western societies report at least intermittent sleep disruptions; up to a quarter suffer from persistent daytime sleepiness, typically as a consequence of insufficient sleep; and 10% experience chronic insomnia.

Sleep disturbances accompany, and causally contribute to, a number of medical, psychiatric, and neurological conditions. Fatigue and insomnia are common symptoms of endocrine and metabolic disorders, such as Addison's disease and the restless leg syndrome of iron deficiency. Inadequate sleep, be it a consequence of lifestyle choices, work or personal pressures, chronic pain, or respiratory dysfunction, is an established risk factor for insulin resistance, diabetes, and obesity. Excessive daytime sleepiness is a major cause of traffic accidents and accident-related deaths.

Complex cause-and-effect relationships characterize sleeping difficulties in psychiatric conditions, especially mood and anxiety disorders. Insomnia and hypersomnia are core symptoms of major depressive disorder, while reduced sleep need is a defining feature of manic episodes. These sleep changes were formerly considered mere epiphenomena to the underlying mood disorders but are now recognized to play aetiological roles: inadequate sleep often triggers episodes, contributes to relapse, and serves as a risk factor for substance abuse comorbidity.

The sleep fragmentation that occurs during normal aging is accelerated or augmented in many neurodegenerative diseases, including Alzheimer's and Parkinson's. Injury to sleep-promoting neurons in the ventrolateral preoptic (VLPO) nucleus of the hypothalamus accounts, in part, for the characteristically poor sleep quality of Alzheimer's patients. In Parkinson's disease, sleep disruptions are among the most diagnostic biomarkers during the prodromal stage and among the most common non-motor signs in symptomatic disease; their severity tends to increase as the disease progresses.

Despite the prevalence of sleep disturbances and the proven benefit of treating them for improving many comorbid conditions, such as depression and chronic pain, therapeutic options remain limited. They include behavioural interventions to improve sleep hygiene and the use of antihistamine and benzodiazepine sedatives. Although among the most commonly prescribed drugs, medications for insomnia carry risks of injury and confusion, morning sedation, anterograde amnesia, and addiction.

There is therefore a need for improved compositions for use in treating sleep disorders, such as insomnia.

It is known that two systems regulate sleep and waking. One of these systems is the circadian clock, which oscillates in synchrony with external changes caused by Earth's rotation. As such, the circadian clock has little to do with the fundamental purpose of sleep: it simply allows animals to schedule their required rest to suit their lifestyles, but it does not explain why sleep is necessary for survival.

The second controller is the sleep homeostat. The homeostat responds to currently ill-defined internal changes that accumulate during waking and directs the reset of the internal changes by vital, but equally ill-defined, functions of sleep. In Drosophila, two dozen sleep-promoting neurons innervating the dorsal fan-shaped body (dFB) of the central complex form the output arm of the sleep homeostat. When the activity of these neurons is impaired, flies are unable to correct sleep deficits and suffer debilitating insomnia. dFB neurons in sleeping flies tend to be electrically active (and by this and other criteria resemble sleep-promoting cells in the VLPO of the mammalian hypothalamus), while neurons in awake flies are electrically silent.

The present invention provides a means to modulate sleep by targeting the process by which sleep-promoting neurons are converted into an electrically active (sleep-inducing) or silent (wake-inducing) state.

Thus, according to a first aspect of the invention, there is provided a ligand of a potassium channel β subunit for use in therapy.

According to another aspect of the invention, there is provided a ligand of a potassium channel β subunit for use in treating or preventing a sleep disorder in a subject.

The potassium channel complex may comprise four α subunits and four β subunits. The α subunits may associate with each other to form a transmembrane pore and the β subunits may associate with each other and the α subunits. The β subunits do not form part of the pore of the channel but regulate its opening and closing.

The four potassium channel α subunits may be referred to as Shaker, which is a potassium channel found in the neurons of Drosophila, particularly dFB neurons. In mammals, potassium channels comprise four α subunits, which may be one or more of the following α subunits: Kv1, Kv2, Kv3, Kv4, Kv5, Kv6, Kv7, Kv8, Kv9, Kv10, Kv11 and Kv12.

The pore of the voltage-gated potassium channel may (temporarily) open in response to depolarisation of the cell membrane, which may be caused by an influx of cations into the cell. When open, the pore allows potassium ions to flow out of the neuron expressing the channel. The flow of potassium ions out of the neuron generates a current, which may be referred to as the A-type potassium current.

The channel may spontaneously inactivate after a brief period of time. While inactivated, the channel cannot be reopened. However, it eventually returns, from a state of inactivation, to a state in which it is capable of being reopened.

The N-terminal domains of the four α subunits may form a hanging platform, suspended below the voltage sensors of the channel, to which the four cytoplasmic β subunits dock. In Drosophila, each of the β subunits may be referred to as Hyperkinetic. The mammalian equivalent to Hyperkinetic may be referred to as K_{V}β (K_{V}β1, K_{V}β2 or K_{B}β3). Hyperkinetic and the orthologous mammalian K_{V}β subunits are related in sequence and structure to aldo-keto-reductases, and may also comprise a catalytic tyrosinate anion, an associated charge-relay system and a nicotinamide cofactor in the active site of the aldo-keto-reductase domain. The nicotinamide cofactor may be nicotinamide adenine dinucleotide phosphate in the reduced form (NADPH) or oxidised form (NADP⁺). Thus, all four of the β subunits may comprise an aldo-keto-reductase domain and a nicotinamide cofactor within the active site of the aldo-keto-reductase domain.

The inventors believe (but do not want to be bound by the theory) that endogenous molecules produced during waking hours induce sleep. More specifically, an endogenous ligand of a potassium channel β subunit is sensed by sleep-promoting neurons (such as dFB neurons of Drosophila and VLPO neurons in mammals). The ligand may be a molecule comprising a carbonyl group (e.g. an aldehyde or a ketone). Such molecules may be produced by lipid peroxidation in sleep-promoting neurons or elsewhere in the body. The molecule may act as a ligand for the aldo-keto-reductase domain of the potassium channel β subunit. The binding of the endogenous ligand to the aldo-keto-reductase domain may result in the oxidation of the nicotinamide cofactor (which may be located in the active site of the aldo-keto-reductase domain) and the reduction of the endogenous ligand per se. The oxidation of the nicotinamide cofactor (e.g. the conversion of NADPH to NADP⁺) may reduce the inactivation of the A-type potassium current. This may increase the rate at which action potentials are fired in dFB neurons, VLPO neurons or other sleep-promoting neurons, which may in turn induce sleep.

Surprisingly, it was found that sleep can be modulated by a ligand of a potassium channel β subunit such as those present in sleep-promoting neurons. Advantageously, therefore, ligands of a potassium channel β subunit can be used to treat a sleep disorder.

Accordingly, in one embodiment, the ligand may be a ligand of a β subunit of a potassium channel. The ligand may be a ligand of the aldo-keto-reductase domain of the β subunit of a potassium channel. The ligand may be a ligand of a β subunit of a potassium channel comprising a Kv1, Kv2, Kv3, Kv4, Kv5, Kv6, Kv7, Kv8, Kv9, Kv10, Kv11 or Kv12 α subunit. Preferably, the ligand is a ligand of a potassium channel comprising a Kv1 α subunit.

Kv1 potassium channel α subunits may be further characterised as Kv1.1, Kv1.2, Kv1.3, Kv1.4, Kv1.5, Kv1.6, Kv1.7 or Kv1.8. Therefore, the ligand may be a ligand of a β subunit of a potassium channel comprising a Kv1.1, Kv1.2, Kv1.3, Kv1.4, Kv1.5, Kv1.6, Kv1.7 or Kv1.8 α subunit. Preferably, the ligand is a ligand of a β subunit of a potassium channel comprising a Kv1.1 α subunit.

The auxiliary β subunits associated with each alpha subunit of the channel may be referred to as Kvβ. Thus, the ligand may be a ligand of Kvβ. The Kvβ subunits may be further characterised as Kvβ1, Kvβ2 or Kvβ3. Thus, the ligand may be a ligand of Kvβ1, Kvβ2 or Kvβ3.

Kvα (α subunits of voltage gated potassium channels) and Kvβ subunits are well known in the art. By way of example only, and in no way intended to be limiting to the invention, the sequence of various Kvα and Kvβ subunits can be found using the following genomic accession numbers:
an accession number of a mRNA sequence encoding human Kv1.1 (KCNA1) is NM_000217;
an accession number of a mRNA sequence encoding Kvβ (KCNAB1) is NM_003471;
an accession number of a mRNA sequence encoding Kvβ2 (KCNAB2) is NM_172130; and
an accession number of a mRNA sequence encoding Kvβ3 (KCNAB3) is NM_172130.

The β subunits may comprise a catalytic tyrosinate anion and an associated charge-relay system. The β subunit of the potassium channel may comprise an aldo-keto-reductase domain and a nicotinamide cofactor (e.g., NADPH or NADP+) within the active site of the aldo-keto reductase domain.

In mammals and Drosophila, the aldo-keto-reductase domain of voltage-gated potassium channel β subunits, such as Hyperkinetic, Kvβ1, Kvβ2, and Kvβ3, may oxidise a nicotinamide cofactor in response to the binding and reduction of a substrate. The aldo-keto-reductase domain of voltage-gated potassium channel β subunits, such as Hyperkinetic, Kvβ1, Kvβ2, and Kvβ3, may reduce a nicotinamide cofactor in response to the binding and oxidation of a substrate.

Thus, the ligand may be a substrate of an aldo-keto-reductase. A substrate may be an agent that binds to the active site of the aldo-keto-reductase domain. The substrate may be an agent that binds to the aldo-keto-reductase domain and induces oxidation or reduction of the nicotinamide cofactor located in the active site of the aldo-keto-reductase domain.

In one embodiment, the substrate may be an electron acceptor, which may be reduced by a potassium channel β subunit, particularly the aldo-keto reductase domain. An electron acceptor, which may be reduced by a potassium channel β subunit, may be referred to as a forward substrate. The forward substrate may be a molecule containing a carbonyl functional group (e.g. an aldehyde or a ketone).

The forward substrate may be a lipid peroxidation product or a derivative thereof. The lipid peroxidation product may be an electron acceptor. The substrate may be a long carbon chain lipid. The substrate may be a long carbon chain lipid containing a carbonyl functional group, such as an aldehyde or a ketone. A long carbon chain may be 6 to 24 consecutive carbons, or 6 to 12 consecutive carbons. Preferably, a long chain carbon comprises 8 to 12 consecutive carbons.

The substrate may be 4-oxo-2-nonenal (4-ONE), 4-hydroxy-2-nonenal (4-HNE), phenylglyoxal, methylglyoxal, 3-deoxyglucosone, 2-carboxybenzaldehyde, 4-carboxybenzaldehyde, 4-cyanobenzaldehyde, acrolein, succinic semialdehyde, (5Z,8Z,10E,14Z)-12-oxoicosa-5,8,10,14-tetraenoic acid (12-oxoETE), prostaglandin J₂, prostaglandin D₂, prostaglandin F_{2α}, 9,10-phenanthrenequinone, 1-palmitoyl-2-(5-oxovaleroyl)-sn-glycero-3-phosphorylcholine (POVPC), 5α-androstan-17β-ol-3-one, or cortisone.

In mammals, voltage-gated potassium channels are expressed in sleep-promoting VLPO neurons, which are considered functionally equivalent to dFB neurons in Drosophila. Thus, administering a ligand according to the invention to a subject will modulate sleep or the propensity to sleep.

In one embodiment, the invention is a forward substrate for use in treating or preventing a sleep disorder in a subject.

In another embodiment, the invention is 4-oxo-2-nonenal (4-ONE) for use in treating or preventing a sleep disorder in a subject. In another embodiment, the invention is 4-hydroxy-2-nonenal (4-HNE) for use in treating or preventing a sleep disorder in a subject.

In one embodiment, the invention is a forward substrate for use in treating or preventing insomnia. The substrate may be used to induce sleep or increase the propensity to sleep in a subject. Thus, the forward substrate may act as a hypnotic in a subject.

In another embodiment, the invention is 4-oxo-2-nonenal (4-ONE) for use in treating or preventing insomnia. In another embodiment, the invention is 4-hydroxy-2-nonenal (4-HNE) for use in treating or preventing insomnia.

In an alternative embodiment, the substrate may be an electron donor, which may be oxidised by a potassium channel β subunit, particularly the aldo-keto-reductase domain. An electron donator, which may be oxidised by a potassium channel τ3 subunit, may be referred to as a reverse substrate. The reverse substrate may be a molecule containing a hydroxyl functional group (e.g. an alcohol).

The reverse substrate may be a long carbon chain lipid. The substrate may be a long carbon chain lipid containing a hydroxyl functional group, such as an alcohol. A long carbon chain may be 6 to 24 consecutive carbons, or 6 to 12 consecutive carbons. Preferably, a long chain carbon comprises 8 to 12 consecutive carbons.

The substrate may be a lipid peroxidation product or a derivative thereof (a reduced lipid peroxidation product).

The substrate may be 4-oxo-2-nonenol or 1,4-dihydroxy-2-nonene. The substrate may be a reduced (alcohol) form of phenylglyoxal, methylglyoxal, 3-deoxyglucosone, 2-carboxybenzaldehyde, 4-carboxybenzaldehyde, 4-cyanobenzaldehyde, acrolein, succinic semialdehyde, (5Z,8Z,10E,14Z)-12-oxoicosa-5,8,10,14-tetraenoic acid (12-oxoETE), prostaglandin J₂, prostaglandin D₂, prostaglandin F_{2α}, 9,10-phenanthrenequinone, 1-palmitoyl-2-(5-oxovaleroyl)-sn-glycero-3-phosphorylcholine (POVPC), 5α-androstan-17β-ol-3-one, or cortisone.

In another embodiment, the invention is 4-oxo-2-nonenol for use in treating or preventing a sleep disorder in a subject. In another embodiment, the invention is 1,4-dihydroxy-2-nonene for use in treating or preventing a sleep disorder in a subject.

In one embodiment, the invention is a reverse substrate for use in treating or preventing narcolepsy. The substrate may be used to prevent sleep or reduce the propensity to sleep in a subject. Thus, the reverse substrate may act as a stimulant in a subject.

In another embodiment, the invention is 4-oxo-2-nonenol for use in treating or preventing narcolepsy. In another embodiment, the invention is 1,4-dihydroxy-2-nonene for use in treating or preventing narcolepsy.

The ligand may be an analogue of a substrate (substrate analogue or competitive inhibitor). The substrate analogue may bind to the active site of the aldo-keto-reductase. The substrate analogue may be a ligand that is not oxidised or reduced. The substrate analogue may be a ligand that is not oxidised or reduced but favours a particular conformation of the aldo-keto-reductase domain, such that sleep or the propensity to sleep is modulated. In the conformation favoured by the substrate analogue, the nicotinamide bound to the active site may be in the oxidised or the reduced form. A substrate analogue may be tolrestat, fidarestat, zopolrestat, sorbinil, caffeic acid phenethyl ester (CAPE), apigenin, luteolin, 7-hydroxyflavone, curcumin, magnolol, honokiol, resveratrol. A competitive inhibitor may bind to the active site of the aldo-keto-reductase domain.

The ligand may be an allosteric ligand. The ligand may bind to the β subunit but may not bind to the active site of the aldo-keto-reductase. The allosteric ligand may favour a particular conformation of the aldo-keto-reductase domain, such that sleep or the propensity to sleep is modulated. In the conformation favoured by the allosteric ligand, the nicotinamide bound to the active site may be in the oxidised or the reduced form.

Preferably, the ligand binds to the active site of the aldo-keto-reductase domain. Preferably, the ligand is hydrophobic so that it is capable of crossing the blood brain barrier.

It will be appreciated that the term “treatment” and “treating” as used herein means the management and care of a subject for the purpose of combating a condition, such as a disease or a disorder. The term is intended to include the full spectrum of treatments for a given condition from which the subject is suffering, including alleviating symptoms or complications, delaying the progression of the disease, disorder or condition, alleviating or relieving the symptoms and complications, and/or to cure or eliminating the disease, disorder or condition as well as to prevent the condition, wherein prevention is to be understood as the management and care of a subject for the purpose of combating the disease, condition, or disorder and includes the administration of the ligand to prevent the onset of the symptoms or complications. The subject to be treated is preferably a mammal, in particular a human, but it may also include animals, such as dogs, cats, horses, cows, sheep and pigs.

A sleep disorder can refer to sleepiness or tiredness during the day. The sleepiness or tiredness during the day may be caused by insufficient sleep, sleep apnea, narcolepsy, restless leg syndrome, drug intake or a medical condition. A sleep disorder can refer to difficulty initiating or maintaining sleep at night (e.g. insomnia), which may result from psychophysiologic causes, inadequate sleep hygiene, psychiatric conditions, medications or drugs of abuse, medical conditions, and neurological conditions. A sleep disorder can refer to unusual behaviours during sleep itself (e.g. parasomnias, which include somnambulism, sleep terrors, sleep bruxism, sleep enuresis, and REM sleep disorder). A sleep disorder can refer to sleepiness or tiredness caused by drug intake (such as the intake of a stimulant or an anaesthetic).

In another aspect of the invention, there is provided a method of treating a subject with a sleep disorder, the method comprising administering a ligand of a potassium channel β subunit to the subject. The ligand may be as described herein.

According to another aspect, there is provided a method of inducing or preventing sleep or modulating the propensity to sleep in a subject, the method comprising administering a ligand of a potassium channel β subunit to the subject. The ligand may be as described herein.

According to another aspect, there is provided a pharmaceutical composition comprising a ligand of a potassium channel β subunit and a pharmaceutically acceptable carrier. The ligand may be as described herein.

Pharmaceutical compositions according to the invention may further comprise a pharmaceutically acceptable salt or other form thereof, together with one or more pharmaceutically acceptable excipients, such as carriers, diluents, fillers, disintegrants, lubricating agents, binders, colorants, pigments, stabilizers, preservatives, antioxidants, and/or solubility enhancers.

The pharmaceutical compositions can be formulated by techniques known in the art. The pharmaceutical compositions can be formulated as dosage forms for oral, parenteral, such as intramuscular, intravenous, subcutaneous, intradermal, intraarterial, intracardial, nasal or aerosol administration. The pharmaceutical composition may be formulated as a dosage form for oral administration.

Dosage forms for oral administration include coated and uncoated tablets, soft gelatin capsules, hard gelatin capsules, lozenges, troches, solutions, emulsions, suspensions, syrups, elixirs, powders and granules for reconstitution, dispersible powders and granules, medicated gums, chewing tablets and effervescent tablets. Dosage forms for parenteral administration include solutions, emulsions, suspensions, dispersions and powders and granules for reconstitution. Emulsions are a preferred dosage form for parenteral administration. Dosage forms for nasal administration can be administered via inhalation and insufflation, for example by a metered inhaler. The ligand or the above-described pharmaceutical compositions may be administered to the subject by any convenient route of administration, whether systemically/peripherally or at the site of desired action including but not limited to one or more of: oral (e.g., as a tablet, capsule, or as an ingestible solution), parenteral (e.g., using injection techniques or infusion techniques, and including, for example, by injection (e.g., subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, or intrasternal by e.g., implant of a depot, for example, subcutaneously or intramuscularly), pulmonary (e.g., by inhalation or insufflation therapy using, e.g., an aerosol, e.g., through mouth or nose), intrauterine, intraocular, intranasal, subcutaneous, ophthalmic (including intravitreal or intracameral).

If the ligand or the pharmaceutical compositions are administered parenterally, then examples of such administration include one or more of: intravenously, intraarterially, intraperitoneally, intrathecally, intraventricularly, intraurethrally, intrasternally, intracardially, intracranially, intramuscularly or subcutaneously, and/or by using infusion techniques. For parenteral administration, the compounds are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known in the art.

The ligand or pharmaceutical compositions can also be administered orally in the form of tablets, capsules, ovules, elixirs, solutions or suspensions, which may contain flavouring or colouring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included. Solid compositions of a similar type may also be employed as fillers in gelatin capsules. Preferred excipients in this regard include lactose, starch, a cellulose, or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

The ligand or pharmaceutical composition may also be administered by sustained release systems. Suitable examples of sustained-release compositions include semi-permeable polymer matrices in the form of shaped articles, e.g., films, or microcapsules. Sustained-release matrices include, e.g., polylactides, copolymers of L-glutamic acid and gamma-ethyl-L-glutamate, poly(2-hydroxyethyl methacrylate), ethylene vinyl acetate or poly-D-(−)-3-hydroxybutyric acid. Sustained-release pharmaceutical compositions also include liposomally entrapped compounds. Liposomes containing a ligand can be prepared by methods known in the art.

Typically, a physician will determine the actual dosage which will be most suitable for an individual subject. The specific dose level and frequency of dosage for any particular individual subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual subject undergoing therapy.

The pharmaceutical composition may contain an excipient to facilitate transport across the blood brain barrier. As used herein, the ‘blood-brain barrier’ or ‘BBB’ refers to the barrier between the peripheral circulation and the brain and the spinal cord which is formed by tight junctions within the brain capillary endothelial plasma membranes, creating an extremely tight barrier that restricts the transport of molecules into the brain. The excipient which facilitates transport across the blood brain barrier refers to a substance that is capable of disrupting or penetrating the blood brain barrier. The amount of excipient administered with the ligand is the amount effective to disrupt the blood brain barrier and allow the ligand to enter the brain.

In a preferred embodiment, the ligand or pharmaceutical composition may be administered by the oral route.

The ligand or pharmaceutical composition may also be administered by the intranasal route. Advantages of the intranasal route include:
unlike parenteral administration, the intranasal administration is not invasive, is generally well tolerated and is easy to self-manage;
unlike what happens after oral administration, the substance administered does not have to pass through the digestive system of the gastrointestinal tract or undergo hepatic metabolization;
he available area of nasal mucosa for absorption is relatively large and easily accessible;
and given that dwell time of the substance in the nose is short, the haematic concentration peak is quickly reached and this can be time by time controlled.

The invention also encompasses non-medical methods of treatment. According to another aspect, there is provided a method of modulating the action potential firing rate of a neuron, the method comprising contacting a neuron with a ligand of a potassium channel β subunit of the neuron.

According to another aspect, there is provided a method of modulating the A-type current of a neuron, the method comprising contacting a neuron with a ligand of a potassium channel β subunit of the neuron.

The neuron may be a sleep promoting-neuron, such as a dFB neuron or a neuron present in the VLPO nuclei.

According to another aspect, there is provided a non-medical method of modulating sleep in a subject, the method comprising administering a ligand of a potassium channel β subunit to the subject.

According to another aspect, there is provided a method of screening a test compound to determine if it is a substrate of a potassium channel β subunit,
the method comprising applying the test compound to a potassium channel β subunit and measuring NADPH oxidation and/or NADP+ reduction,
wherein if NADPH is oxidised or NADP⁺ is reduced after applying the test compound, the test compound is a substrate.

If NADPH is oxidised after applying the test compound, the test compound may be a forward substrate. If NADP⁺ is reduced after applying the test compound, the test compound may be a reverse substrate.

According to another aspect, there is provided a method of screening a test compound to determine if it is a substrate of a potassium channel β subunit,
the method comprising applying the test compound to a cell comprising a potassium channel having a β subunit,
inducing at least two action potentials within the cell, and
measuring the firing rate of the action potentials within the cell,
wherein if the firing rate of the action potentials increases or decreases after applying the test compound, the test compound is a substrate,

If the firing rate of the action potentials increases after applying the test compound, the test compound may be a forward substrate. If the firing rate of the action potentials decreases after applying the test compound, the test compound may be a reverse substrate.

According to another aspect, there is provided a method of screening a test compound to determine if it is a substrate of a potassium channel β subunit,
the method comprising applying the test compound to a cell comprising a potassium channel having a β subunit, and
measuring the A-type potassium current,
wherein if inactivation of the A-type potassium current is slowed or accelerated after applying the test compound, the compound is a substrate.

If inactivation of the A-type potassium current is slowed after applying the test compound, the test compound may be a forward substrate. If inactivation of the A-type potassium current is accelerated after applying the test compound, the test compound may be a reverse substrate.

According to another aspect, there is provided a method of screening a test compound to determine if it is a substrate of a potassium channel β subunit,
the method comprising administering the test compound to an organism and measuring sleep,
wherein if sleep increases or decreases after administering the test compound, the compound is a substrate.

If sleep increases after administering the test compound, the test compound may be a forward substrate. If sleep decreases after administering the test compound, the test compound may be a reverse substrate.

The skilled person would appreciate that ligands of a potassium channel β subunit may be identified by various assays, including a biochemical assay, an electrophysiological assay and a behavioural assay.

A biochemical assay may be an enzymatic assay that can be used to verify the effect of a test compound on the aldo-keto-reductase activity of Hyperkinetic or a mammalian Kvβ ortholog. The biochemical assay may comprise incubating a test compound with purified Kvβ protein bound to NADPH, and then measuring NADPH absorption using a UV spectrometer. Any changes in NADPH absorption over time reflect the oxidation of NADPH to NADP⁺ and the coupled reduction of the test compound by the aldo-keto-reductase. Thus, any such changes in absorption indicate that the test substrate is an electron acceptor in the active site of the aldo-keto-reductase. The reverse reaction (i.e. the oxidation of alcohols to aldehydes or ketones, which is coupled to the reduction of NADP⁺ to NADPH) may be tested by incubating NADP⁺-bound Kvβ with a test compound. Any changes in NADP⁺ absorption over time reflect the reduction of NADP⁺ to NADPH and the coupled oxidation of the test compound by the aldo-keto-reductase. Thus, any such changes indicate that the test compound is an electron donor in the active site of the aldo-keto-reductase. The assay may be performed using a catalytically inactive Kvβ protein (in parallel) in order to confirm that any changes in absorption, which may be observed, are in fact due to the catalytic activity of the aldo-keto-reductase.

An electrophysiological assay may be a whole-cell patch-clamp assay. The patch-clamp assay may be used to verify if a test compound modulates an A-type current of a cell expressing Shaker or a mammalian orthologue and Hyperkinetic or a mammalian Kvβ orthologue. The assay may comprise transfecting cultured cells, such as human embryonic kidney cells 293 (HEK293), with Hyperkinetic (or an orthologue thereof) and the voltage-gated potassium channel Shaker (or an orthologue thereof) and performing whole-cell patch-clamp experiments. Current or voltage step protocols may be applied to the cell in order to extract the action potential firing rate or A-type potassium current, respectively, in the presence or absence of the test compound. An increase in the action potential firing rate is indicative that the compound is a forward substrate of the aldo-keto-reductase, whereas a decrease in the action potential firing rate is indicative that the compound is a reverse substrate of the aldo-keto-reductase. An increase or decrease in the A-type current may be indicative that the test compound is a substrate of the aldo-keto-reductase. The whole-cell patch-clamp assay may be performed using a catalytically inactive Hyperkinetic protein or an orthologue thereof (in parallel) in order to confirm that any changes in the action potential firing rate or the A-type current, which may occur, are in fact mediated via the active site of the aldo-keto-reductase.

A behavioural assay may be an assay that can be used to verify the effect of a test compound on sleep. The assay may comprise placing individual 3-5 days old female flies (D. melanogaster) in separate 65 mm long glass tubes with food at one end and a cotton plug at the other end, and exposing the tubes to 12 h light:12 h dark conditions. The activity of the flies may then be measured using the Trikinetics Drosophila Activity Monitor system (TriKinetics Inc., Walham, Mass., USA). Periods of inactivity lasting at least 5 minutes are classified as sleep episodes. The sleep- or wake-promoting effects of the test compound may be tested by adding the compound to the food of the flies. Multiple sleep parameters, such as total amount of daytime sleep, total amount of night-time sleep, average sleep episode length, number of sleep episodes, and others may then be quantified. Potential effects on locomotion may be assessed by measuring waking activity. An increase in the duration of sleep, the average length of each sleep episode, or the number of sleep episodes may be indicative that the test compound is a forward substrate in the active site of the aldo-keto-reductase, whereas a decrease in the duration of sleep, the average length of each sleep episode, or the number of sleep episodes may be indicative that the test compound is a reverse substrate in the active site of the aldo-keto-reductase. To ensure that the compounds act via Hyperkinetic, wild-type flies and Hyperkinetic catalytic mutant or knockdown flies may be tested in parallel.

All of the features described herein (including any accompanying claims, abstract and drawings), and/or all of the steps of any method or process so disclosed, may be combined with any of the above aspects or embodiments in any combination, except combinations where at least some of such features and/or steps are mutually exclusive.

For a better understanding of the invention, and to show how embodiments of the same may be carried into effect, reference will now be made, by way of example, to the accompanying Figures, in which:—

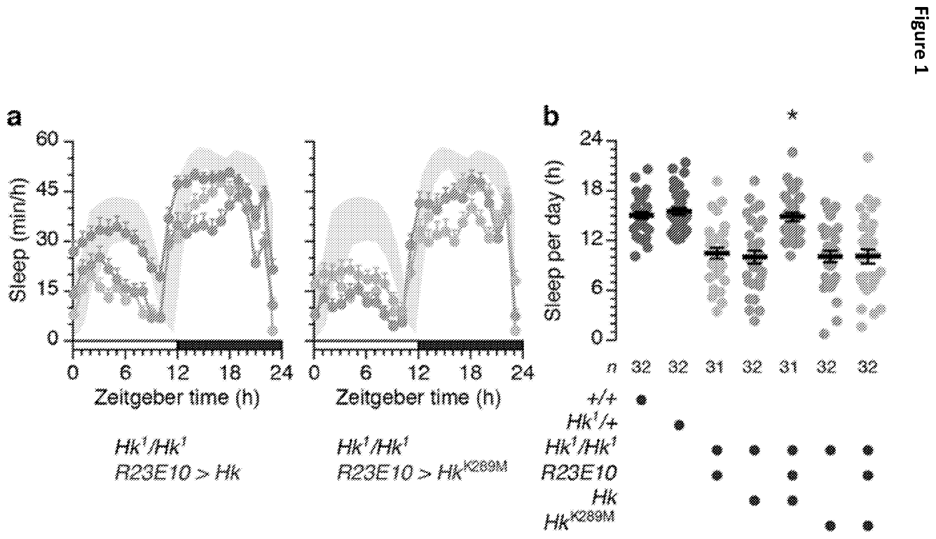

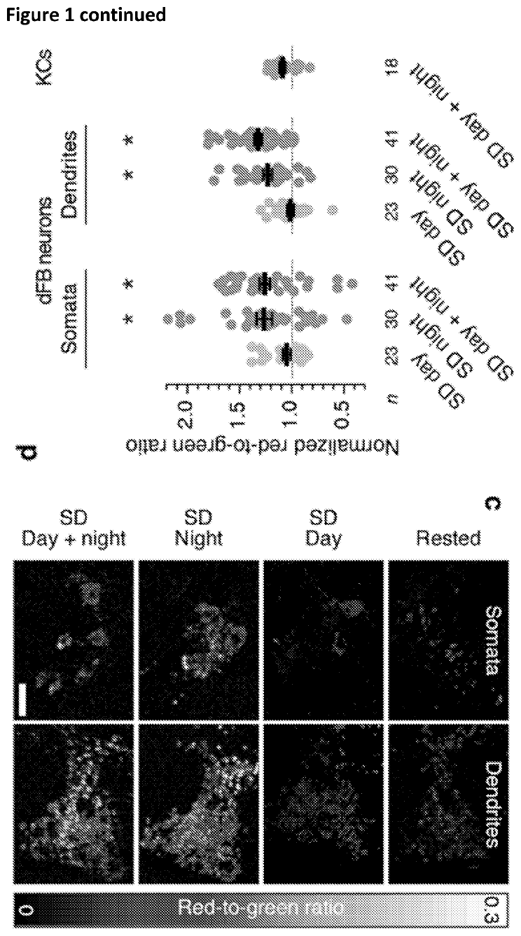

FIG. 1—Hyperkinetic senses redox changes linked to sleep history. a, R23E10-GAL4-driven expression of Hk (Hyperkinetic) (black, left), but not of a catalytically inactive variant (Hk^{K289M}, black, right), in a homozygous Hk¹ mutant background elevates sleep relative to parental controls (grey colours as in b), to wild-type level (shaded bands: 95% confidence intervals). Data are means±s.e.m.; sample sizes are reported in b. Two-way repeated-measures ANOVA detected significant differences from both parental controls (P<0.0001) but not from wild-type (P=0.9973) in flies expressing Hk, and a significant difference from wild-type (P<0.0001) but not from either parental control (P>0.9833) in flies expressing Hk^{K289M}. b, Sleep in homozygous Hk¹ mutants expressing R23E10-GAL4-driven Hk rescue transgenes and parental, wild-type, and heterozygous controls (circles: individual flies; bars: means s.e.m.). One-way ANOVA detected significant differences from both parental controls (P<0.0001) but not from wild-type (P=0.9763) in flies expressing Hk, and a significant difference from wild-type (P<0.0001) but not from either parental control (P>0.9704) in flies expressing Hk^{K289M}; the asterisk indicates a significant difference from both parental controls in pairwise post-hoc comparisons. c, Maximum intensity projections of the somata and dendritic arbors of dFB neurons expressing MitoTimer under R23E10-GAL4 control, in rested and sleep-deprived (SD) flies. The ratio of fluorescence emissions at 571 and 525 nm is pseudocoloured according to the key on the right. Scale bar, 10 μm. d, Sleep deprivation during the night, but not during the day (P>0.6416, Mann-Whitney test), increases MitoTimer's red-to-green ratio in somata and dendrites of dFB neurons (P<0.0001, Kruskal-Wallis ANOVA) but not Kenyon cell (KC) dendrites (P=0.1328, t test); asterisks indicate significant differences from rested conditions in pairwise post-hoc comparisons. Fluorescence ratios are normalized to those of unperturbed controls at the end of sleep deprivation (n=22 and 62 dFB controls for daytime and night-time deprivation; n=20 KC controls);

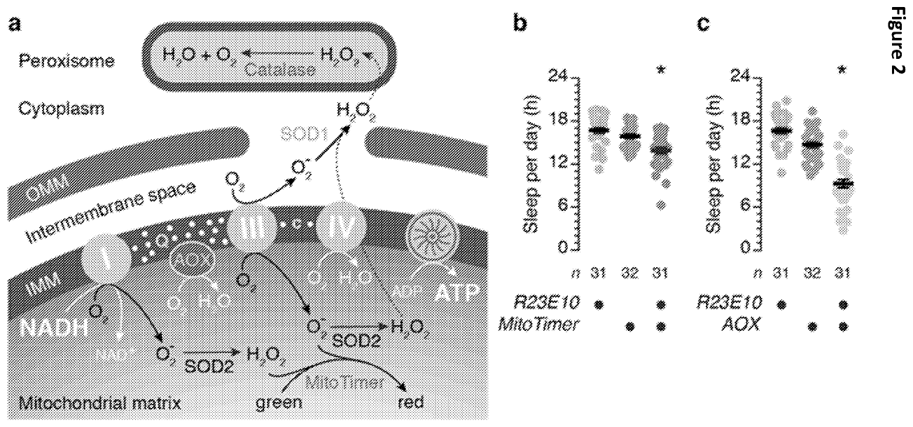

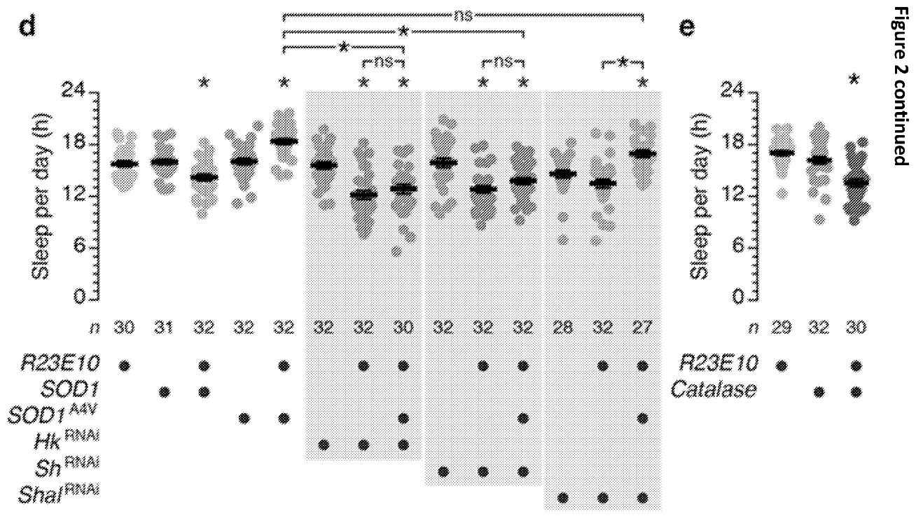

FIG. 2—dFB-restricted perturbations of redox chemistry alter sleep. a, Ubiquinone (Q) and cytochrome c (c) ferry electrons (white dots) between the proton-pumping complexes I, III, and IV of the mitochondrial transport chain. When more electrons enter the chain than can be used to fuel ATP synthesis (that is, when NADH is abundant, the proton-motive force is large, and/or ATP demand is low), a backlog of electrons accumulate in the Q pool between complexes I and III. These electrons react directly with O₂, releasing O₂⁻ into the matrix and the space between the inner and outer mitochondrial membranes (IMM and OMM). Superoxide dismutases (SOD2 in the matrix, SOD1 in the intermembrane space and cytoplasm) convert O₂⁻ to membrane-permeant H₂O₂; catalase decomposes H₂O₂ further. AOX, a terminal oxidase not present in most animals, uses surplus Q electrons to reduce O₂ to water. b, Sleep in flies expressing R23E10-GAL4-driven MitoTimer and parental controls (circles: individual flies; bars: means±s.e.m.). One-way ANOVA detected a significant genotype effect (P<0.0001); the asterisk indicates a significant difference from both parental controls in pairwise post-hoc comparisons. c, Sleep in flies expressing R23E10-GAL4-driven AOX and parental controls (circles: individual flies; bars: means±s.e.m.). One-way ANOVA detected a significant genotype effect (P<0.0001); the asterisk indicates a significant difference from both parental controls in pairwise post-hoc comparisons. d, Sleep in flies expressing R23E10-GAL4-driven SOD1 or a pro-oxidant variant (SOD1^{A4V}), with or without RNAi transgenes targeting K_{V} channel subunits, and parental controls (circles: individual flies; bars: means s.e.m.). One-way ANOVA detected a significant genotype effect (P<0.0001); asterisks indicate significant differences from parental controls or in relevant pairwise post-hoc comparisons (brackets). e, Sleep in flies expressing R23E10-GAL4-driven catalase and parental controls (circles: individual flies; bars: means±s.e.m.). One-way ANOVA detected a significant genotype effect (P<0.0001); the asterisk indicates a significant difference from both parental controls in pairwise post-hoc comparisons;

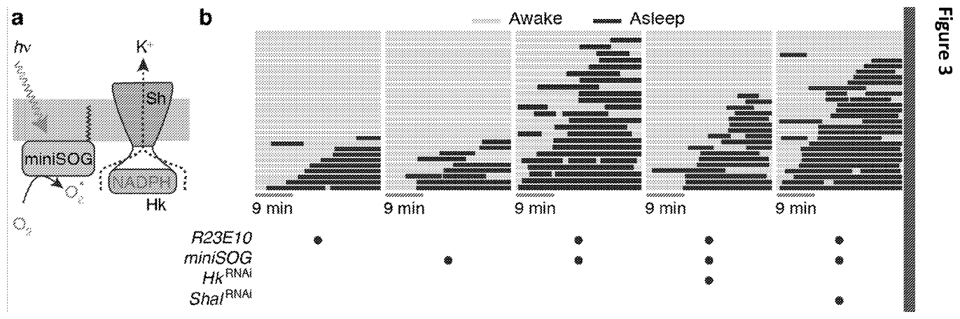

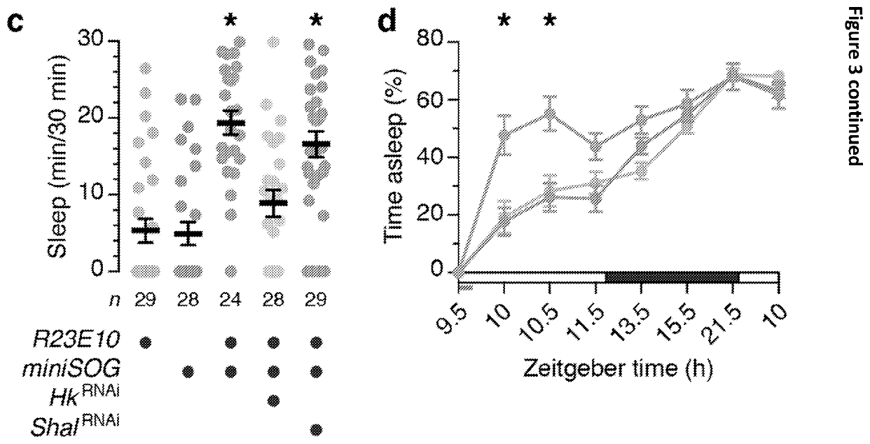

FIG. 3—Optogenetically controlled ROS production in dFB neurons induces sleep. a, An N-myristoyl group anchors miniSOG at the cytoplasmic face of the plasma membrane, near the Hyperkinetic (Hk) gondola suspended beneath Shaker (Sh). b, Periods of wake (gray) and sleep (black) during and after an initial 9-min exposure to blue light, in flies expressing R23E10-GAL4-driven miniSOG, with or without RNAi transgenes targeting K_{V} channel subunits, and parental controls. Each row depicts one individual; all individuals were awake at the onset of illumination. The fraction of experimental flies falling asleep differed from both parental controls (P<0.0001, χ² test with pairwise post-hoc comparisons) and from flies coexpressing Hk^{RNAi} but not Shal^{RNAi} (P=0.0030, χ² test). c, Sleep in flies expressing R23E10-GAL4-driven miniSOG, with or without RNAi transgenes targeting K_{V} channel subunits, and parental controls (circles: individual flies; bars: means±s.e.m.). Kruskal-Wallis ANOVA detected a significant genotype effect (P<0.0001); asterisks indicate significant differences from controls in pairwise post-hoc comparisons. d, Cumulative sleep percentages at different time points after a 9-min exposure to blue light at zeitgeber time 9.5 h (means±s.e.m.), in flies expressing R23E10-GAL4-driven miniSOG (n=19, black) and parental controls (n=25 each, gray colours as in c). Two-way repeated-measures ANOVA detected a significant time×genotype interaction (P<0.0001); asterisks indicate time points when sleep differed significantly between experimental flies and both parental controls;

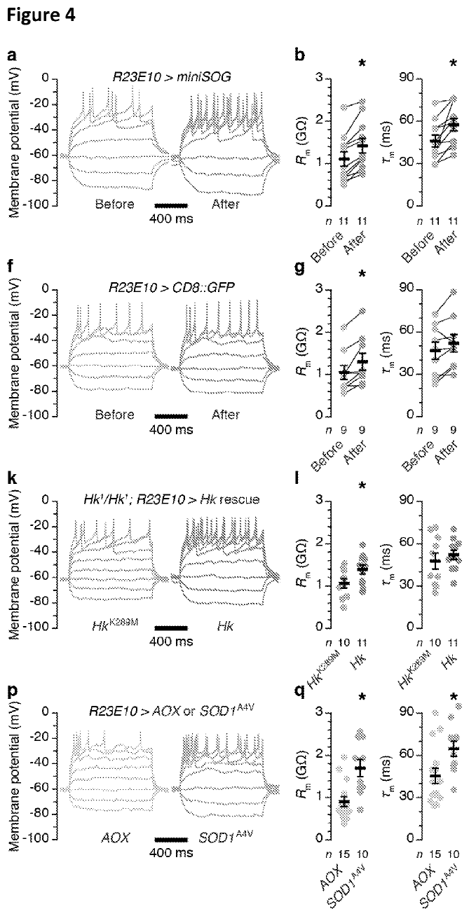

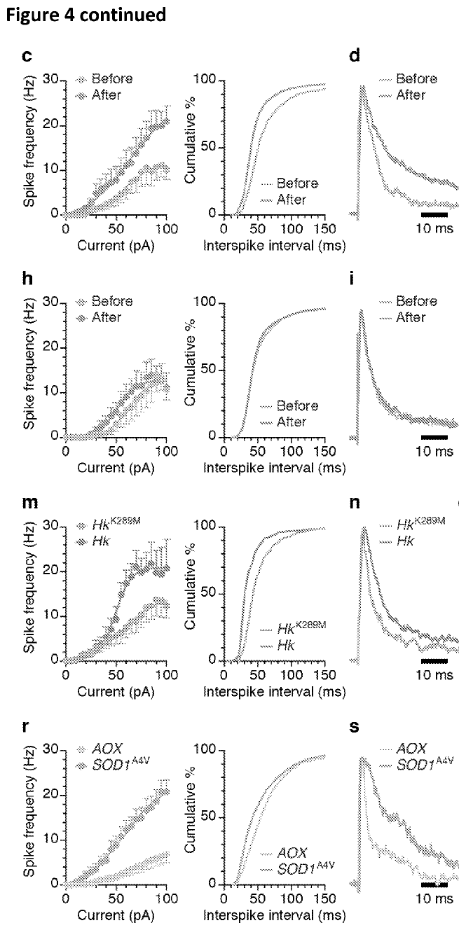

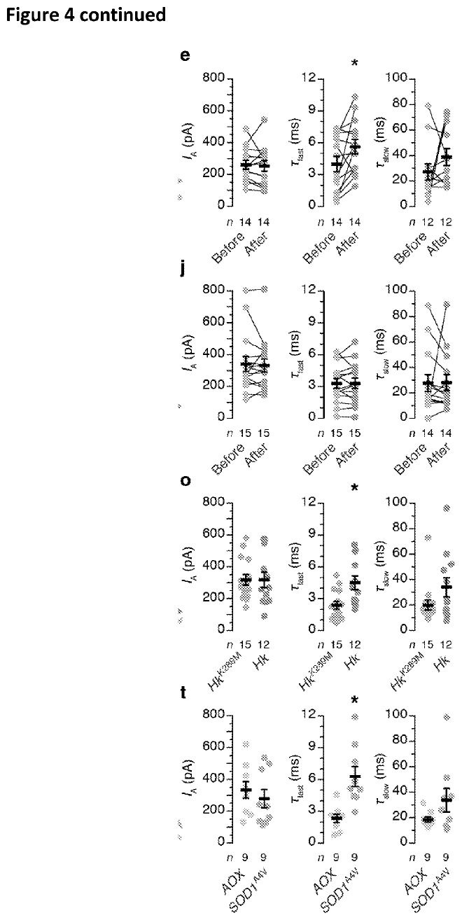

FIG. 4—Changes in redox chemistry alter the electrical activity of dFB neurons via I_{A}. a-e, dFB neurons expressing R23E10-GAL4-driven miniSOG and CD8::GFP, before and after a 9-min exposure to blue light. Voltage responses to current steps (a): illumination increases the input resistance (b, Rₘ; P<0.0001, paired t test) and membrane time constant (b, τₘ; P=0.0041, paired t test), steepens the current-spike frequency function (c, left; P=0.0014, two-way repeated-measures ANOVA), and shifts the interspike interval distribution toward shorter values (c, right; P<0.0001, Kolmogorov-Smirnov test). I_{A} (normalized to peak) evoked by voltage steps to +40 mV (d): illumination leaves the I_{A} amplitude unchanged (e; P=0.7295, paired t test) and increases the fast (e, τ_{fast}; P=0.0245, Wilcoxon test) but not the slow inactivation time constant (e, τ_{slow}; P=0.3804, Wilcoxon test). f-j, dFB neurons expressing R23E10-GAL4-driven CD8::GFP, before and after a 9-min exposure to blue light. Voltage responses to current steps (f): illumination increases the input resistance (g, Rₘ; P=0.0098, paired t test) but not the membrane time constant (g, τₘ; P=0.0723, paired t test) and leaves unchanged the current-spike frequency function (h, left; P=0.9982, two-way repeated-measures ANOVA) and interspike interval distribution (h, right; P=0.0947, Kolmogorov-Smirnov test). I_{A} (normalized to peak) evoked by voltage steps to +40 mV (i): illumination leaves unchanged the I_{A} amplitude (j; P=0.8040, Wilcoxon test) and both inactivation time constants (j, τ_{fast}: P=0.6387, τ_{slow}: P=0.2958, Wilcoxon tests). k-o, dFB neurons expressing R23E10-GAL4-driven Hk^{K2S9M} or Hk rescue transgenes in a homozygous Hk¹ mutant background. Voltage responses to current steps (k): the restoration of functional Hk increases the input resistance (l, Rₘ; P=0.0467, t test) but not the membrane time constant (l, τₘ; P=0.4962, t test), steepens the current-spike frequency function (m, left; P<0.0001, two-way repeated-measures ANOVA), and shifts the interspike interval distribution toward shorter values (m, right; P<0.0001, Kolmogorov-Smirnov test). I_{A} (normalized to peak) evoked by voltage steps to +40 mV (n): the restoration of functional Hk leaves the I_{A} amplitude unchanged (o; P=0.9827, t test) and increases the fast (o, τ_{fast}, P=0.0061, t test) but not the slow inactivation time constant (o, τ_{slow}; P=0.1257, Mann-Whitney test). p-t, dFB neurons expressing R23E10-GAL4-driven AOX or SOD1^{A4V}. Voltage responses to current steps (p): the expression of pro-oxidant SOD1^{A4V} increases the input resistance (q, Rₘ; P=0.0023, Mann-Whitney test) and membrane time constant (q, τₘ; P=0.0166, Mann-Whitney test), steepens the current-spike frequency function (r, left; P<0.0001, two-way repeated-measures ANOVA), and shifts the interspike interval distribution toward shorter values (r, right; P<0.0001, Kolmogorov-Smirnov test). I_{A} (normalized to peak) evoked by voltage steps to +40 mV (s): the expression of pro-oxidant SOD1^{A4V} leaves the I_{A} amplitude unchanged (t; P=0.4892, t test) and increases the fast (t, τ_{fast}; P=0.0013, t test) but not the slow inactivation time constant (t, τ_{slow}; P=0.3401, Mann-Whitney test);

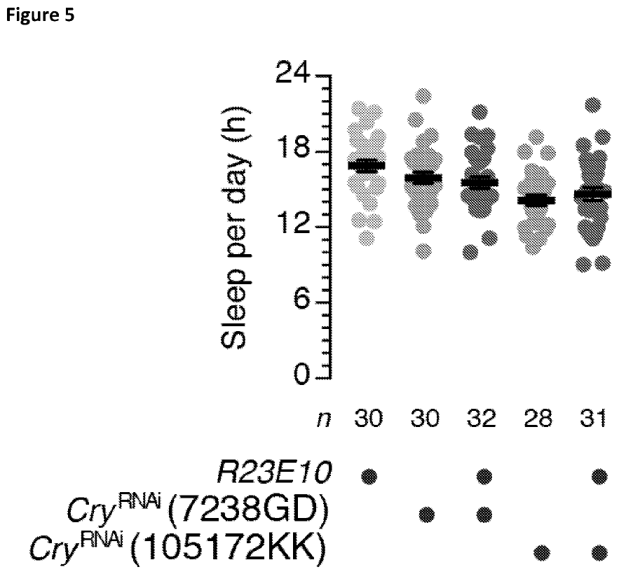

FIG. 5—Chronic dFB-restricted perturbations of cryptochrome have no impact on sleep. Sleep in flies expressing two different R23E10-GAL4-driven cry^{RNAi} transgenes and parental controls (circles: individual flies; bars: means±s.e.m.). One-way ANOVA failed to detect significant differences of experimental flies from both of their respective parental controls (P>0.1718);

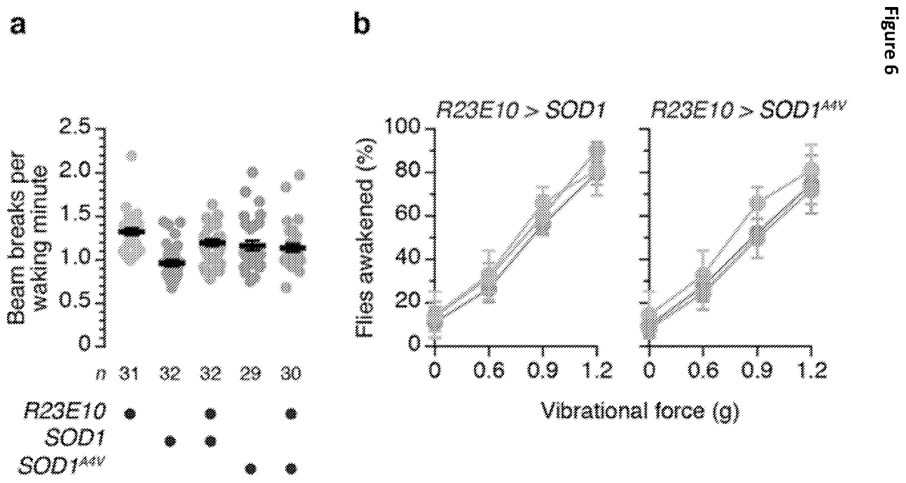

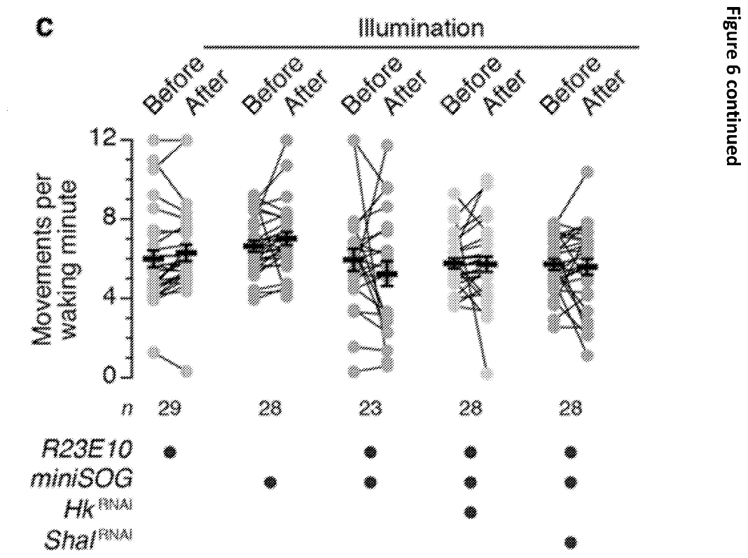

FIG. 6—Chronic or acute dFB-restricted perturbations of redox chemistry have no impact on waking locomotor activity or arousability. a, Locomotor counts per waking minute of flies expressing R23E10-GAL4-driven SOD1 or a pro-oxidant variant (SOD1^{A4V}), in the Trikinetics Drosophila Activity Monitor system. Kruskal-Wallis ANOVA failed to detect significant differences of experimental flies from both of their respective parental controls (P>0.2612). b, Arousability of flies expressing R23E10-GAL4-driven SOD1 (black, left) or a pro-oxidant variant (SOD1^{A4V}, black, right) and parental controls (gray colours as in a). Data are means±s.e.m. of 6 trials per genotype (n=16-32 flies each). Two-way ANOVA detected a significant effect of vibrational force (P<0.0001) but not of genotype (P>0.2487). c, Locomotor counts per waking minute of flies expressing R23E10-GAL4-driven miniSOG, with or without RNAi transgenes targeting K_{V} channel subunits, and parental controls, in a custom video-tracking system¹⁵. Activity was monitored for 10 min before the photooxidation of miniSOG and then for a 30-min interval that included a 9-min exposure to blue light. Two-way repeated-measures ANOVA failed to detect significant effects of genotype (P=0.0827) and illumination (P=0.8059) and a significant interaction between the two factors (P=0.3086); and

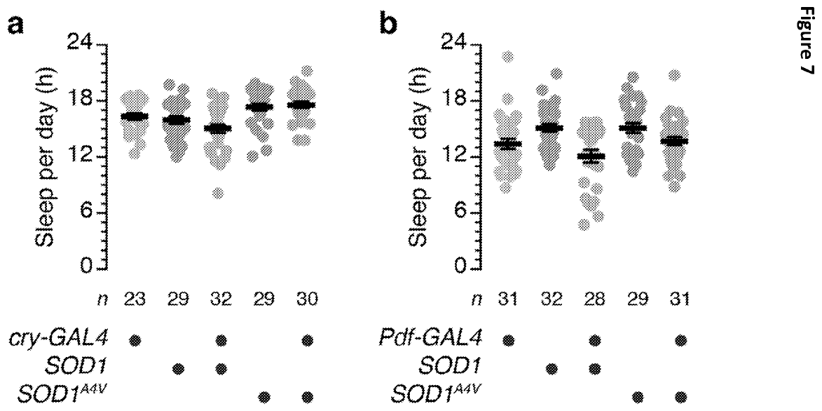

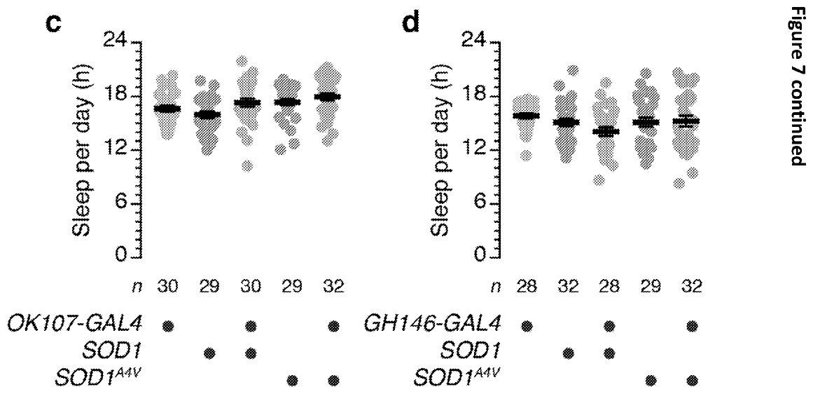

FIG. 7—Chronic perturbations of redox chemistry in cryptochrome- or Pdf-expressing clock neurons, Kenyon cells, or olfactory projection neurons have no impact on sleep. a, Sleep in flies expressing cry-GAL4-driven SOD1 or pro-oxidant variant (SOD1^{A4V}) in clock neurons and parental controls (circles: individual flies; bars: means±s.e.m.). Kruskal-Wallis ANOVA failed to detect significant differences of experimental flies from both of their respective parental controls (P>0.1426). b, Sleep in flies expressing Pdf-GAL4-driven SOD1 or a pro-oxidant variant (SOD1^{A4V}) in clock neurons and parental controls (circles: individual flies; bars: means±s.e.m.). Kruskal-Wallis ANOVA failed to detect significant differences of experimental flies from both of their respective parental controls (P>0.1732). c, Sleep in flies expressing OK107-GAL4-driven SOD1 or a pro-oxidant variant (SOD1^{A4V}) in Kenyon cells and parental controls (circles: individual flies; bars: means±s.e.m.). One-way ANOVA failed to detect significant differences of experimental flies from both of their respective parental controls (P>0.0603). d, Sleep in flies expressing GH146-GAL4-driven SOD1 or a pro-oxidant variant (SOD1^{A4V}) in olfactory projection neurons and parental controls (circles: individual flies; bars: means±s.e.m.). Kruskal-Wallis ANOVA failed to detect significant differences of experimental flies from both of their respective parental controls (P>0.6901).

### EXAMPLES

### Methods

Drosophila strains and culture. Fly stocks were grown on media of sucrose, yeast, molasses, and agar under a 12 h light: 12 h dark cycle at 25° C. All studies were performed on females aged 2-6 days post eclosion. Experimental flies were heterozygous for all transgenes and homozygous for either a wild-type or mutant (Hk¹) Hyperkinetic allele, as indicated. Driver lines R23E10-GAL4, cry-GAL4, pdf-GAL4, OK107-GAL4, and GH146-GAL4 were used to target dFB neurons, cryptochrome- or PDF-expressing clock neurons, Kenyon cells, or olfactory projection neurons, respectively. Effector transgenes encoded a fluorescent marker for visually guided patch-clamp recordings (UAS-CD8::GFP); wild-type or mutant (Hk^{K289M}) Hyperkinetic rescue transgenes; an optical integrator of ROS exposure in the mitochondrial matrix (UAS-MitoTimer); the mitochondrial alternative oxidase AOX; wild-type and mutant (SOD1^{A4V}) versions of human superoxide dismutase 1; catalase; an N-myristoylated covalent hexamer (myr-MS6T2) of the singlet oxygen generator miniSOG; and RNAi constructs for interference with the expression of Hyperkinetic, Shaker, Shal, or cryptochrome (101402KK, 104474KK, 103363KK, and 7238GD or 105172KK, respectively; Vienna Drosophila Resource Center).

Sleep measurements. In standard sleep assays, females aged 3-5 d were individually inserted into 65-mm glass tubes, loaded into the Trikinetics Drosophila Activity Monitor system, and housed under 12 h light: 12 h dark conditions. Periods of inactivity lasting at least 5 minutes were classified as sleep. Immobile flies (<2 beam breaks per 24 h) were excluded from the analysis. In sleep deprivation experiments, a spring-loaded platform stacked with Trikinetics monitors was slowly tilted by an electric motor, released, and allowed to snap back to its original position². The mechanical cycles lasted 12 s and were repeated continuously.

Arousal thresholds in standard sleep assays were determined with the help of mechanical stimuli generated by vibration motors (Precision Microdrives, model 310-113). Stimuli were delivered for 15 s, once every hour, and the percentages of sleeping flies awakened during each stimulation episode were quantified.

Sleep after light-induced ROS generation was measured at zeitgeber time 9.5 h. Female flies aged 3-5 d and expressing miniSOG in dFB neurons were individually inserted into 35-mm glass tubes and loaded into a custom-built array of light-tight chambers. Each chamber was equipped with a high-power LED (Osram Opto Semiconductors LB W5SM-FZHX-35-0, 467 nm) running at an 80% duty cycle at 10 Hz and delivering 8 mW cm⁻² at the distal and 80 mW cm⁻² at the proximal end of the tube. In this intensity range, each miniSOG molecule in the central brain underwent an estimated 2-40 excitation cycles s⁻¹, based on the measured optical transmission of 7 fly heads at 467 nm (4.8±0.3% (mean+s.e.m.), assumed to be isotropic) and a miniSOG absorption cross-section⁴⁶ of 5.0×10⁻¹⁷ cm⁻².

The apparatus was operated in a temperature-controlled incubator (Sanyo MIR-154) at 25° C. Excess heat from the high-power LEDs was removed by a water-cooling device incorporating liquid heat exchangers (Thermo Electric Devices LI102), a centrifugal pump (RS 702-6891), Peltier module (Adaptive ETC-128-10-05-E), and CPU cooler (Corsair CW-9060007-WW). For movement tracking, the chambers were continuously illuminated by low-power infrared (850 nm) LEDs from below and imaged from above at 25 frames with a high-resolution CMOS camera (Thorlabs DCC1545M), using an 8 mm lens (Thorlabs MVL8M23) and a long-pass filter (Thorlabs, FEL800 nm) to reject photostimulation light. A virtual instrument written in LabVIEW (National Instruments) extracted real-time position data from video images by subtracting the most recently acquired image from a temporally low-pass-filtered background. Non-zero pixels in the difference image indicated that a movement had occurred, with the centroid of the largest cluster of non-zero pixels taken to represent the fly's new position. To eliminate noise, intensity and size thresholds were applied to pixel clusters in the difference image, and movements<2.5 mm (approximately one body length) were discarded. Periods of inactivity lasting at least 5 minutes were classified as sleep. The flies were monitored for 10 min before the photooxidation of miniSOG, and subjects found asleep during that period were excluded from the analysis. Only individuals with a confirmed waking time>30 s were used to quantify waking movements, which were counted as distinct events if they were separated by >5 s of immobility.

Functional imaging. Single-housed females were analysed 2-6 days post eclosion after 12 or 24 h of mechanical sleep deprivation, begun at zeitgeber times 0 h (daytime deprivation) or 12 h (night-time deprivation), and compared to age-matched controls at the end of sleep deprivation. After head-fixing the flies to a custom mount with eicosane (Sigma), cuticle, adipose tissue, and trachea were removed to create a small surgical window, and the brain was continuously superfused with extracellular solution equilibrated with 95% O₂-5% CO₂ and containing 103 mM NaCl, 3 mM KCl, 5 mM TES, 8 mM trehalose, 10 mM glucose, 7 mM sucrose, 26 mM NaHCO₃, 1 mM NaH₂PO₄, 1.5 mM CaCl₂, 4 mM MgCl₂, pH 7.3.

MitoTimer fluorescence was imaged in vivo by two-photon laser-scanning microscopy. Excitation light pulses with 140 fs duration and a centre wavelength of 910 nm (Chameleon Ultra II, Coherent) were intensity-modulated with the help of a Pockels cell (302RM, Conoptics) and focused by a 20><, 1.0 NA water immersion objective (W-Plan-Apochromat, Zeiss) on a Movable Objective Microscope (Sutter Instruments). Emitted photons were separated from excitation light by a series of dichromatic mirrors and dielectric and coloured glass filters, split into red and green channels (Semrock BrightLine FF01-571/72 and FF01-525/45, respectively), and detected by GaAsP photomultiplier tubes (H10770PA-40 SEL, Hamamatsu Photonics). Photocurrents were passed through high-speed amplifiers (HCA-4M-500K-C, Laser Components) and custom-designed integrator circuits to maximize the signal-to-noise ratio. The microscope was controlled through ScanImage (Vidrio Technologies) via a PCI-6110 DAQ board (National Instruments). Images were acquired as z-stacks with an axial resolution of 1 μm.

Maximum-intensity projections of image stacks were analysed blind to sleep history, using a semi-automated script in MATLAB (The MathWorks). The algorithm rejected saturated or MitoTimer-negative pixels (fluorescence<1.5-fold above the mean of a manually defined background area) and calculated the average red-to-green ratio for the remaining image area.

Electrophysiology. For whole-cell patch-clamp recordings in vivo, female flies aged 3-5 days post eclosion were prepared as for functional imaging, but the perineural sheath was also removed for electrode access. The somata of GFP-labeled dFB neurons were visually targeted with borosilicate glass electrodes (12-14 mil). The internal solution contained 140 mM potassium aspartate, 10 mM HEPES, 1 mM KCl, 4 mM MgATP, 0.5 mM Na₃GTP, 1 mM EGTA, pH 7.3. Signals were acquired with a Multiclamp 700B amplifier (Molecular Devices), filtered at 6-10 kHz, and digitised at 10-20 kHz using an ITC-18 data acquisition board (InstruTECH) controlled by the Nclamp/Neuromatic package. Data were analysed using Neuromatic software (www.neuromatic.thinkrandom.com) and custom procedures in Igor Pro (Wavemetrics).

For photostimulation of miniSOG during whole-cell recordings, a 455-nm LED

(Thorlabs M455L3) was focused onto the head of the fly with a mounted f=20.1 mm aspheric condenser lens (Thorlabs ACP2520-A) and controlled by a TTL-triggered dimmable constant-current LED driver (Thorlabs LEDD1B). The optical power at the sample was ˜3.5 mW cm⁻².

Membrane resistances were calculated from linear fits of the steady-state voltage changes elicited by 1-s steps of hyperpolarizing currents (5-pA increments) from a pre-pulse potential of −60±5 mV. Membrane time constants were estimated by fitting a single exponential to the voltage deflection caused by a hyperpolarizing 10-pA current step lasting 200 ms. Interspike intervals were determined from voltage responses to a standard series of depolarizing current steps (5 pA increments from 0 to 100 pA, 1 s duration). Spikes were detected by finding minima in the time derivative of the membrane potential trace. Interspike intervals at all levels of injected current were pooled for the calculation of frequency distributions.

Voltage-clamp experiments were performed in the presence of 1 μM tetrodotoxin (Tocris) and 200 μM cadmium to block sodium and calcium channels, respectively. Neurons were stepped from holding potentials of −110 or −30 mV to a test potential of +40 mV. When the cells were held at −110 mV, depolarization steps (1 s duration) elicited the full complement of potassium currents; when the cells were held at −30 mV, voltage-gated channels inactivated and the evoked potassium currents lacked the I_{A} (A-type or fast outward) component. Digital subtraction of the non-A-type component from the full complement of potassium currents gave an estimate of I_{A}. To determine the fast and slow inactivation time constants, double exponential functions were fit to the decaying phase of currents elicited by 1-s depolarizing voltage pulses after digitally subtracting non-inactivating outward currents (Table 1). In cases where the fits of slow inactivation time constants were poorly constrained, only the fast inactivation time constants were included in the analysis.

Statistics. Data were analysed in Prism 7 (GraphPad). Group means were compared by one-way or two-way ANOVA, using repeated measures designs where appropriate, followed by planned pairwise post hoc analyses using Holm-Šídák's multiple comparisons test. Where the assumptions of normality or sphericity were violated (as indicated by Shapiro-Wilk and Brown-Forsythe tests, respectively), group means were compared by two-sided Mann-Whitney, Wilcoxon, or Kruskal-Wallis tests; the latter was followed by Dunn's multiple comparisons test. χ² tests were performed on contingency tables of categorical data. Interspike interval distributions were evaluated by Kolmogorov-Smirnov test. The investigators were blind to group allocation in MitoTimer imaging experiments but not otherwise. No statistical methods were used to predetermine sample sizes.

### Example 1—Redox Regulation of Sleep Via K_{V}β

Mutations in Shaker or Hyperkinetic both cause insomnia. Unsurprisingly, given the importance of A-type currents for sustaining the sleep-promoting activity of these cells, dFB neurons are a major sleep-relevant site of action for both potassium channel subunits: the depletion of either gene product from these cells alone, using R23E10-GAL4-restricted RNA interference (RNAi), reproduces the sleep disruptions of the genomic mutations. To complement these demonstrations of necessity with a test of sufficiency, Hyperkinetic expression was exclusively restored in the dFB of otherwise homozygous mutant flies. Sleep returned to wild-type levels, but only if Hyperkinetic's active site was intact (FIG. 1a, b): a putative rescue transgene encoding a variant with a point mutation (K289M) that abolishes the protein's oxidoreductase activity but leaves its expression and the amplitude of I_{A} unaltered (see later) proved ineffective. This finding has three implications. First, it suggests that Hyperkinetic's sleep-regulatory role in dFB neurons is tied to its ability to sense changes in intracellular redox state. Since dFB neurons convey the homeostatic response to sleep loss, redox changes are therefore expected to accompany changes in sleep pressure. Second, it predicts that perturbing the redox chemistry of dFB neurons will have consequences for sleep. And third, it identifies a biophysical mechanism for coupling redox chemistry and sleep. Because redox reactions, oxygen use, and ATP synthesis are linked at the level of the flow of reducing equivalents through the mitochondrial electron transport chain, dFB neurons may monitor redox processes as a gauge of energy metabolism. Established relationships of caloric intake, oxidative stress, and sleep to senescence and degenerative disease may therefore have a common basis.

### Example 2—Metabolic Origin of Sleep Pressure

To examine the first implication of Hyperkinetic's obligatory catalytic competence, the redox histories of flies that had been mechanically sleep-deprived was compared with those of rested controls (FIG. 1c, d). The metabolic machinery in the inner mitochondrial membrane and matrix is the principal cellular source of oxidants, especially under conditions of ample NADH supply, large proton-motive force, and low ATP demand, when electrons stall in the transport chain and transfer directly to oxygen, producing superoxide (O₂⁻) that is subsequently dismuted to H₂O₂ (FIG. 2a). Chief conduits for electron leakage are a fully reduced ubiquinone pool and the resulting tailback of electrons onto the flavin mononucleotide cofactor of Complex I. Although some ROS produced in the mitochondrial electron transport chain could conceivably reach the active site of Hyperkinetic by diffusion, a more plausible scenario is that O₂⁻ and H₂O₂ react locally and release a longer-lived carbonyl substrate whose reduction by Hyperkinetic then causes the oxidation of NADPH. Lipid peroxidation products, such as the aldehyde 4-oxo-2-nonenal, serve as established hydride acceptors in K_{V}β subunits and may represent the ill-defined electron densities overlying their hydrophobic active sites.

To obtain a cumulative estimate of mitochondrial ROS production, the mitochondria of dFB neurons was labelled with a matrix-targeted fluorescent protein (MitoTimer) whose green-emitting chromophore converts irreversibly to red when oxidized. Age-matched flies were then deprived of variable amounts of sleep and the ratio of red to green emissions was determined by two-photon microscopy. Mitochondrial ROS production rose roughly in proportion to the size of the imposed sleep deficits: a night of sleep deprivation red-shifted MitoTimer's fluorescence relative to rested controls, but applying the same sleep deprivation protocol during the day, when flies are naturally awake, or adding a day to a night of sleep disruption produced only insignificant effects (FIG. 1c, d). Because dFB neurons generate few energetically costly action potentials in the awake, fed state, when calories are plentiful but the Sandman detent blocks spiking, the condition of a high ATP:ADP ratio known to favour mitochondrial O₂⁻ production in the presence of a continuous supply of reducing substrates is likely to be met. Consistent with this idea, mushroom body Kenyon cells, which are electrically active during waking, showed little evidence of heightened oxidant exposure even after 24 h of sleep deprivation (FIG. 1d). In addition, or instead, dFB neurons may have an unusually low capacity for degrading ROS, making them canaries in the mine for their detection.

Curiously, flies expressing MitoTimer in dFB neurons lost ˜2 h of baseline sleep per day compared to parental controls (FIG. 2b). As the oxidation of MitoTimer will consume ROS, this finding was interpreted as tentative evidence of a causal connection between mitochondrial oxidative burden and sleep. To strengthen this connection, sleep was quantified after three further dFB-neuron-specific interventions: manipulation of mitochondrial electron transport; chronic interference with antioxidant enzymes; and acute optogenetic induction of singlet oxygen (¹O₂) formation in the vicinity of the Shaker-Hyperkinetic complex.

An electron overflow pathway was first installed in the inner mitochondrial membrane of dFB neurons by expressing the alternative oxidase AOX of Ciona intestinalis. Like Complex III, AOX taps into the ubiquinone pool, but instead of transferring an electron each to two cytochrome c carriers (and in the process pumping two protons across the inner membrane), it reduces molecular oxygen to water in a single four-electron transfer reaction (FIG. 2a). Alternative respiration thus siphons off electrons that would otherwise spill from the ubiquinone pool and produce ROS when the cytochrome branch of the transport chain is saturated or the availability of ADP is low. Introducing AOX into the mitochondria of dFB neurons, which normally lack a capacity for alternative respiration, decreased daily sleep by nearly 7 h (FIG. 2c). Clamping mitochondrial ROS production thus eased the pressure to sleep.

In animals without bifurcated electron transport chains, superoxide dismutases (SODs) and catalase, which acts as a sink for SOD-generated H₂O₂ and thereby also pulls the dismutation reaction forward, form the first line of anti-oxidant defense (FIG. 2a). Shoring up these defenses by overexpressing SOD1 or catalase in dFB neurons reduced sleep (FIG. 2d, e), while breaching them with the help of a mutant enzyme (SOD1^{A4V}) whose peroxidase activity is enhanced due to inadequate shielding of the catalytic copper ion had the converse effect; it increased sleep (FIG. 2d) without inhibiting waking locomotion (FIG. 6a) or arousability (FIG. 6b). The crucial link between changes in redox chemistry and sleep was the Shaker-Hyperkinetic complex: the RNAi-mediated depletion of either channel subunit from dFB neurons not only occluded the sleep-promoting effect of SOD1^{A4V} but reduced sleep below wild-type levels (FIG. 2d). In contrast, interference with the expression of Shal, a K_{V} channel without a sleep-regulatory function in dFB neurons, proved innocuous (FIG. 2d).

Analogous SOD1 manipulations in cryptochrome- or PDF-positive clock neurons or Kenyon cells (which all have demonstrated roles in sleep control) or in olfactory projection neurons (for which no such role has been reported) failed to influence sleep (FIG. 7a-d) dFB neurons thus appear unique, at least among this comparison group, in their ability to transduce oxidative stress into sleep.

As a third test of the redox control of sleep, miniSOG, an engineered flavoprotein that photogenerates ¹O₂, was anchored via a myristoyl group at the cytoplasmic face of the plasma membrane (FIG. 3a). If the light-driven release of ¹O₂ near Hyperkinetic causes the oxidation of bound NADPH, either directly or via local lipid peroxidation, it should be possible to bypass the entire chain of metabolic events that couples this final transduction step to mitochondrial respiration and induce sleep acutely. To determine whether this was the case, sleep was monitored for a 30-min period that included an initial 9-min exposure to blue light. Flies expressing miniSOG in dFB neurons fell quiescent in greater proportion, and for longer, than control flies did (FIG. 3b, c). Epochs of quiescence outlasted the illumination period by ˜1 h (FIG. 3d), could be blocked by the removal of Hyperkinetic but not of Shal (FIG. 3b, c), and were not due to the suppression of waking movements (FIG. 6c).

Example 3—Transduction of sleep pressure into sleep Whole-cell recordings from dFB neurons in vivo, before and after miniSOG-mediated photooxidation under similar sleep-inducing conditions, revealed some of the well-documented biophysical changes underpinning the wake-sleep switch: the neurons' action potential responses to depolarizing current became more vigorous (FIG. 4a-c); their membrane time constants lengthened (FIG. 4b); the interspike interval contracted (FIG. 4a, c); and the fast inactivation rate of their A-type potassium currents slowed (FIG. 4d, e, Table 1). Changes in repetitive firing and the inactivation kinetics of I_{A} are, of course, mechanistically connected. Both are regulated by K_{V}β subunits, with oxidation of NADPH to NADP⁺, slow inactivation, and high-frequency activity typically going hand in hand. A-type channels in the conducting state constitute the repolarizing that returns the membrane potential to its resting level after a spike; reducing their rate of inactivation therefore accelerates the release of the next action potential and so enables tonically active neurons to fire at higher rates.

Like the induction of sleep (FIG. 3b, c), these biophysical changes required the abrupt burst of ROS production caused by the high ¹O₂ quantum yield of miniSOG. No cell physiological changes—apart from a modest increase in input resistance—were seen after equally intense and prolonged irradiation of dFB neurons expressing membrane-bound GFP, whose chromophore is encased in a protein shell that prevents the close apposition of O₂ necessary for efficient energy transfer (FIG. 4f-j).

TABLE 1
Parameters of I_{A} inactivation.
Time constants were obtained by fitting double exponential
functions to the decaying phase of I_{A} in dFB neurons, evoked
by voltage steps to +40 mV. A_{fast}/(A_{fast} + A_{slow)}
represents the fraction of the fast component of the total A-current.
Data are means ± s.e.m.; n indicates the number of cells. All neurons
express CD8::GFP in addition to the indicated transgene.

| | τ_{fast} (ms) | τ_{slow} (MS) | A_{fast}/(A_{fast} + A_{slow}) |
|---|---|---|---|
| R23E10 > miniSOG | | | |
| before illumination | 4.01 ± 0.72 | 27.18 ± 6.39 | 0.58 ± 0.07 |
| after illumination | 5.64 ± 0.67 | 38.91 ± 6.57 | 0.63 ± 0.06 |
| n | 14 | 12 | 12 |
| R23E10 > CD8::GFP | | | |
| before illumination | 3.31 ± 0.47 | 27.85 ± 6.65 | 0.65 ± 0.05 |
| after illumination | 3.32 ± 0.50 | 28.22 ± 6.13 | 0.56 ± 0.05 |
| n | 15 | 14 | 14 |
| R23E10 > Hk^{K289M} | | | |
| n | 2.40 ± 0.36 | 19.87 ± 3.98 | 0.50 ± 0.04 |
| | 15 | 15 | 15 |
| R23E10 > Hk | | | |
| n | 4.50 ± 0.65 | 33.94 ± 7.52 | 0.67 ± 0.04 |
| | 12 | 12 | 12 |
| R23E10 > AOX | | | |
| n | 2.35 ± 0.38 | 18.58 ± 1.99 | 0.54 ± 0.04 |
| | 9 | 9 | 9 |
| R23E10 > SOD1^{A4V} | | | |
| n | 6.30 ± 0.94 | 33.89 ± 9.32 | 0.73 ± 0.07 |
| | 9 | 9 | 9 |

The coherent picture emerging from these within-cell analyses was mirrored in between-cell comparisons of neurons with chronically altered redox-sensing or redox-buffering capacity: the homozygous Hyperkinetic mutants carrying catalytically active or dead rescue transgenes that were our point of departure (FIG. 4k-o), or cells containing pro-oxidant SOD1^{A4V} or anti-oxidant AOX (FIG. 4p-t). dFB neurons equipped with a functional Shaker β subunit expressed slowly inactivating A-type currents (FIG. 4n, o) that enabled high-frequency action potential trains⁴⁸ (FIG. 4k, m). In flies forced to make do with the K289M mutant, which cannot convert NADPH to NADP⁺, dFB neurons exhibited fast-inactivating I_{A} (FIG. 4n, o), long interspike intervals (FIG. 4k, m), and shallow current-spike frequency functions (FIG. 4k, m) that can account for the insomnia of these animals (FIG. 1a, b). Profound shifts of Hyperkinetic's NADP⁺:NADPH ratio in opposite directions must also underlie the divergent interspike interval distributions (FIG. 4p, r), current-spike frequency functions (FIG. 4p, r), and A-type inactivation kinetics of dFB neurons expressing SOD1^{A4V} or AOX (FIG. 4s, t), which parallel large and opposite changes in daily sleep (FIG. 2c, d).

### Example 4—Accumulation and Discharge of Sleep Pressure

Because K_{V}β subunits have very low cofactor exchange rates that limit their enzymatic turnover, perhaps to a single hydride transfer, even a fleeting exposure of the permanently bound cofactor to an oxidant will form a lasting biochemical memory. The Shaker-Hyperkinetic complex therefore unites three discrete functions in a single device: Its redox sensitivity allows it to monitor a key process relevant to sleep—the generation of oxidative by-products of mitochondrial electron transport. Its catalytic inefficiency allows the protein to compute and store the time integral of the resulting oxidative burden, as would be required if sleep's purpose were to protect against oxidative stress. And its ability to set the spike frequency via conformational coupling to the channel's inactivation gate allows it to titrate the commensurate corrective action.

The molecular interpretation of sleep pressure as a progressive conversion of Hyperkinetic to the NADP⁺-bound form immediately suggests how this process could be influenced pharmacologically. Depending on their redox potential relative to the bound cofactor species, small-molecule K_{V}β substrates will collect electrons from NADPH or lose them to NADP⁺ and in this manner alter the NADP⁺:NADPH ratio. Electron acceptors, like the aldehydes produced endogenously during the breakdown of lipid peroxides, are predicted to exert hypnotic effects through the coupled oxidation of NADPH, while the corresponding alcohols should act as stimulants by reducing NADP⁺.

In order to dissipate the accumulated sleep pressure, the NADP⁺:NADPH ratio must return to baseline during sleep. An elegant way to accomplish this reset would be to gate Hyperkinetic's enzymatic activity by voltage. Cofactor release from the active site would be impeded in fill mode because the membrane potential of dFB neurons remains below the activation threshold of Shaker, but in discharge mode, when the neurons fire action potentials, the voltage-driven rearrangements of the channel would open an escape route for NADP⁺. Bidirectional coupling of a redox-modulated ion channel and a voltage-modulated oxidoreductase may thus be the accounting principle at the heart of the somnostat.

1. A ligand of a potassium channel β subunit for use in therapy.

2. A ligand of a potassium channel β subunit for use in treating or preventing a sleep disorder in a subject.

3. The ligand for use according to claim 1 or claim 2, wherein the ligand is a ligand of an aldo-keto-reductase domain of the β subunit of a potassium channel.

4. The ligand for use according to any preceding claim, wherein the ligand is a ligand of a (3 subunit of a potassium channel comprising a Kv1, Kv2, Kv3, Kv4, Kv5, Kv6, Kv7, Kv8, Kv9, Kv10, Kv11 or Kv12 α subunit.

5. The ligand for use according to any preceding claim, wherein the ligand is a ligand of a β subunit of a potassium channel comprising a Kv1.1, Kv1.2, Kv1.3, Kv1.4, Kv1.5, Kv1.6, Kv1.7 or Kv1.8 α subunit.

6. The ligand for use according to any preceding claim, wherein the ligand is a ligand of a Kvβ1, Kvβ2 or Kvβ3 subunit.

7. The ligand for use according to any preceding claim, wherein the ligand does not bind to the aldo-keto-reductase domain.

8. The ligand for use according to any preceding claim, wherein the ligand binds to the active site of the aldo-keto-reductase domain.

9. The ligand for use according to any preceding claim, wherein the ligand is an allosteric ligand.

10. The ligand for use according to claim 3, wherein the ligand is a substrate of the aldo-keto-reductase.

11. The ligand for use according to claim 10, wherein the substrate is an electron acceptor.

12. The ligand for use according to claim 10 or 11, wherein the substrate contains a carbonyl functional group (e.g. an aldehyde or a ketone).

13. The ligand for use according to any one of claims 10 to 12, wherein the substrate is 4-oxo-2-nonenal (4-ONE), 4-hydroxy-2-nonenal (4-HNE) phenylglyoxal, methylglyoxal, 3-deoxyglucosone, 2-carboxybenzaldehyde, 4-carboxybenzaldehyde, 4-cyanobenzaldehyde, acrolein, succinic semialdehyde, (5Z,8Z,10E,14Z)-12-oxoicosa-5,8,10,14-tetraenoic acid (12-oxoETE), prostaglandin J₂, prostaglandin D₂, prostaglandin F₂—, 9,10-phenanthrenequinone, 1-palmitoyl-2-(5-oxovaleroyl)-sn-glycero-3-phosphorylcholine (POVPC), 5α-androstan-17β-ol-3-one, or cortisone.

14. The ligand for use according to any preceding claim, wherein the sleep disorder is insomnia, sleep apnea, restless leg syndrome, drug intake or a medical, neurological or psychiatric condition.

15. The ligand for use according to claim 10, wherein the substrate is an electron donor.

16. The ligand for use according to claim 10 or claim 15, wherein the substrate comprises a hydroxyl functional group (e.g. an alcohol).

17. The ligand for use according to claim 10, claim 15 or claim 16, wherein the substrate is 4-oxo-2-nonenol, 1,4-dihydroxy-2-nonene or a reduced (alcohol) form of a selection from the group comprising/consisting of: phenylglyoxal, methylglyoxal, 3-deoxyglucosone, 2-carboxybenzaldehyde, 4-carboxybenzaldehyde, 4-cyanobenzaldehyde, acrolein, succinic semialdehyde, (5Z,8Z,10E,14Z)-12-oxoicosa-5,8,10,14-tetraenoic acid (12-oxoETE), prostaglandin J₂, prostaglandin D₂, prostaglandin F_{2α}, 9,10-phenanthrenequinone, 1-palmitoyl-2-(5-oxovaleroyl)-sn-glycero-3-phosphorylcholine (POVPC), 5α-androstan-17β-ol-3-one, or cortisone.

18. The ligand for use according to any one of claims 1 to 10 or claims 15 to 17, wherein the sleep disorder is narcolepsy or a medical, neurological or psychiatric condition.

19. A method of treating a subject with a sleep disorder, the method comprising administering a ligand of a potassium channel β subunit to the subject.

20. A pharmaceutical composition comprising a ligand of a potassium channel β subunit and a pharmaceutically acceptable carrier.

21. A method of modulating the action potential firing rate of a neuron, the method comprising contacting a neuron with a ligand of a potassium channel β subunit of the neuron.

22. A method of modulating the A-type current of a neuron, the method comprising contacting a neuron with a ligand of a potassium channel β subunit of the neuron.

23. The method according to claim 21 or claim 22, wherein the neuron is a sleep promoting-neuron, such as a dFB neuron or a neuron present in the VLPO nuclei.

24. The method of claim 19, 21, 22 or 23 or the pharmaceutical composition of claim 20 wherein the ligand is as defined in any of claims 3 to 13 or 15 to 17.

25. A method of screening a test compound to determine if it is a substrate of a potassium channel β subunit, the method comprising
applying the test compound to a potassium channel β subunit, and
measuring NADPH oxidation and/or NADP+ reduction,
wherein if NADPH is oxidised or NADP⁺ is reduced after applying of the test compound, the test compound is a substrate.

26. The method of claim 15, wherein if NADPH is oxidised after applying the test compound, the test compound is a forward substrate, and wherein if NADP⁺ is reduced after applying the test compound, the test compound is a reverse substrate.

27. A method of screening a test compound to determine if it is a substrate of a potassium channel β subunit, the method comprising applying the test compound to a cell comprising a potassium channel having a β subunit,
inducing at least two action potentials within the cell, and
measuring the firing rate of the action potentials within the cell,
wherein if the firing rate of the action potentials increases or decreases after applying the test compound, the test compound is a substrate,

28. The method of claim 27, wherein if the firing rate of the action potentials increases after applying the test compound, the test compound is a forward substrate, and if the firing rate of the action potentials decreases after applying of the test compound, the test compound is a reverse substrate.

29. A method of screening a test compound to determine if it is a substrate of a potassium channel β subunit, the method comprising
applying the test compound to a cell comprising a potassium channel having a β subunit, and
measuring the A-type potassium current,
wherein if inactivation of the A-type potassium current is slowed or accelerated after applying the test compound, the compound is a substrate.

30. The method of claim 29, wherein if inactivation of the A-type potassium current is slowed after applying the test compound, the test compound is a forward substrate, and wherein if inactivation of the A-type potassium current is accelerated after applying the test compound, the test compound is a reverse substrate.

31. A method of screening a test compound to determine if it is a substrate of a potassium channel β subunit, the method comprising
administering the test compound to an organism, and
measuring sleep,
wherein if sleep increases or decreases after administering the test compound, the compound is a substrate.

32. The method of claim 31, wherein if sleep increases after administering the test compound, the test compound is a forward substrate, and wherein if sleep decreases after administering the test compound, the test compound is a reverse substrate.

33. The method of claim 31 or claim 32, wherein an increase in sleep is an increase in the duration of sleep, the average length of each sleep episode, or the number of sleep episodes.

34. The method of any one of claims 31 to 33, wherein a decrease in sleep is a decrease in the duration of sleep, the average length of each sleep episode, or the number of sleep episodes.
